(19) United States
(12) Patent Application Publication
(10) Pub. No.: US 2004/0097957 A1
(43) Pub. Date: May 20, 2004

(54) Medical device, drug delivery and lab sampling system utilizing an inverting sheath technology

(76) Inventors: Marc Jaker, New Brighton, MN (US); Mark Nelson, St. Paul, MN (US)

Correspondence Address: SNELL & WILMER, ONE ARIZONA CENTER, 400 EAST VAN BUREN, PHOENIX, AZ, 850040001

(21) Appl. No.: 10/689,826

(22) Filed: Oct. 20, 2003

Related U.S. Application Data
(60) Provisional application No. 60/419,758, filed on Oct. 18, 2002.

(51) Int. Cl. A61F 9/00

(52) U.S. Cl. 606/107

(57) ABSTRACT

An inverting sheath delivery system for medical devices, intraocular lens (IOL), and stent placement, in an eye as part of a cataract procedure or other body location or for therapeutic procedures such as the precise placement of radioisotopes (seeds) to a tumor. In one embodiment, the IOL replacement is positioned in the sheath and carried forward as the sheath is advanced during inversion. The IOL is then deposited in the corresponding juxtaposed position proportional to the distance the sheath traveled around the tip of the pusher tube.

### CROSS-REFERENCE TO RELATED APPLICATIONS

This application claims priority to, and the benefit of, U.S. Provisional Patent Application Serial No.: 60/419,758, entitled “MEDICAL DEVICE, DRUG DELIVERY AND LAB SAMPLING SYSTEM UTILIZING AN INVERTING SHEATH TECHNOLOGY” and filed Oct. 18, 2002.

### TECHNICAL FIELD

The present invention generally relates to medical introducers, delivery tools and systems used for medical procedures. In particular, the present invention relates to the placement of a medical device (e.g., stent, intraocular ophthalmic lens, small implantable devices, and the like) and to the placement and positioning of radiologic and other drugs, for example, isotopes used to treat tumors, cancerous tissue, unwanted growths, fungi and the like.

### BACKGROUND INFORMATION

Delivery tools for medical devices and therapeutic drugs are nearly as old as medicine itself. However, the more recent implantable devices such as coronary, prostate, and esophageal stents1, ophthalmic lens replacements, heart valve replacements and the expanded drug treatment of deep tumors, growths, and infections have put new demands on these delivery systems.

Delivery systems for medical devices typically demand lubrication. A common problem for these delivery systems is infections, with some systems having a higher infection rate than other systems. Many factors influence the infection rate including the patient's age and health, and the anatomical location where the procedure is being performed.

Current delivery systems for stents typically use a rigid polymer introducer/catheter through which the stent is placed on an inflatable angio balloon structure for positioning and expansion at the site (e.g., vascular, urethral, esophageal, etc.). Ophthalmic lens replacement (or prosthetic) typically use a semi-rigid polymer mini tube through which a precisely lubricated and rolled (compressed) lens is pushed into position within the eye with a plunger.

In addition, drug therapies are generally introduced through rigid catheter tubes, for example, stainless steel tubes used during chemotherapy.

Pathology sampling is typically taken via swabs or similar devices which pass through a body orifice to the site, thus risking possible contamination from areas in the path, for example, the urethra meatus in urology or the mouth in throat/esophageal cultures and thus cross contaminating the swab.

The present invention addresses these long felt needs from a new perspective using inverting sheath technology.

### SUMMARY OF THE INVENTION

In accordance with one aspect of the present invention, a polymer sheath delivery system for medical devices and therapeutic drugs includes a sheath that inverts back and forth to engulf and load medical devices and therapeutic drugs. The inverting membrane sheath may be made of a fluoropolymer resin or fluoro-alloy such as polytetrafluoroethylene (PTFE), for example as may be used in other Memcath™ devices and products or similarly performing polymer materials. The membrane sheath may deploy the medical device or drug to the desired position in the body where the medical device or drug was inserted, positioned, and enveloped into the membrane sheath. For example, three centimeters from the leading edge into the sheath equals three centimeters depth into the body.

In accordance with another aspect of the present invention, a lay flat self-collapsing sheath is provided that collapses and protects the medical device such as an intraocular lens (IOL) or a stent, as the sheath is pulled into the pusher/deployment/positioning tube. The inverting sheath deployment delivery system has the versatility to accommodate multiple medical applications with dimensional changes, to accommodate various diameters and lengths.

In accordance with another aspect of the present invention, the sheath will advance with a sampling reagent, and the sampling reagent will then retreat back into the sheath, thus providing a completely sterile, localized bacterial sampling for pathology diagnostic use.

In accordance with another aspect of the present invention, lubricant is not delivered and lubricant does not remain in the eye during IOL delivery. In addition, contaminants from the conjunctiva, urethra, or other body part or channel are not dragged into the wound during introduction of a medical device.

In accordance with another aspect of the present invention, a syringe type deployment system is provided to facilitate the sheath/device loading and deployment.

In accordance with another aspect of the present invention, an easily loaded device that utilizes a lay flat polymer sheath is provided that will not damage pre-set devices such as stents or an IOL.

The invention herein described is intended to facilitate the placement and/or positioning of smaller medical devices and therapeutic drugs, in a clean non-lubricated fashion. The sheath may collapse IOL's and stents as they are drawn down into its inside diameter just prior to a procedure thus avoiding complicated device pre-prep and keeping devices material IOL (silicone or acrylic) or stents (Nitonol) from taking on a set or welding between similar materials.

### BRIEF DESCRIPTION OF THE DRAWINGS

A more complete understanding of the present invention may be derived by referring to the detailed description when considered in connection with the Figures, wherein like reference numbers refer to similar elements throughout the Figures, and:

FIG. 1 is a perspective view of a membrane sheath in accordance with one embodiment of the present invention;

FIG. 2 is a perspective view of various aspects of a delivery assembly in accordance with an embodiment of the present invention;

FIG. 3 is a side view of various aspects of a delivery assembly in accordance with an embodiment of the present invention;

FIG. 4 is a side view of the delivery assembly of FIG. 3 during a first aspect of a delivery operation;

FIG. 5 is a side view of the delivery assembly of FIG. 3 during a further aspect of a delivery operation;

FIG. 6 is a side view of a delivery assembly in accordance with an alternative embodiment of the present invention;

FIG. 7 is a side view of the delivery assembly of FIG. 6 during a first aspect of a delivery operation;

FIG. 8 is a side view of the delivery assembly of FIG. 6 during a further aspect of a delivery operation;

FIG. 9 is a side view of a delivery assembly in accordance with a further alternative embodiment of the present invention;

FIG. 10 is a side view of the delivery assembly of FIG. 9 during a first aspect of a delivery operation;

FIG. 11 is a side view of the delivery assembly of FIG. 9 during a further aspect of a delivery operation;

FIG. 12 is a side view of a delivery assembly in accordance with a further alternative embodiment of the present invention;

FIG. 13 is a side view of various exemplary pusher tube tips in accordance with various aspects of the present invention;

FIG. 14 is a side view and a front view of an alternate tapered pusher tube tip in accordance with the present invention;

FIG. 15 is a side view of an alternative embodiment of a pusher tube in accordance with the present invention;

FIG. 16 is a perspective view of various aspects of an alternative embodiment of a delivery assembly in accordance with the present invention;

FIG. 17 is a side view of a delivery assembly in accordance with a further alternative embodiment of the present invention during a first aspect of a delivery operation;

FIG. 18 is a side view of the delivery assembly of FIG. 17 during a further aspect of a delivery operation;

FIG. 19 is a side view of a delivery tube in accordance with a further alternative embodiment of the present invention;

FIG. 20 is a side view of the delivery tube of FIG. 19 showing the two tube sections;

FIG. 21 is a perspective view of one end of the delivery tube of FIG. 19;

FIG. 22 is a side view of a delivery assembly using the delivery tube of FIG. 19 during a first aspect of a delivery operation;

FIG. 23 is a side view of the delivery assembly of FIG. 22 during a further aspect of a delivery operation; and

FIG. 24 is a top view of the delivery assembly of FIG. 22 during a further aspect of a delivery operation.

### DETAILED DESCRIPTION OF EXEMPLARY EMBODIMENTS

The present invention may be described herein in terms of various hardware components and modules and processing steps. It should be appreciated that such modules and steps may be realized by any number of hardware components configured to perform the specified functions. For example, the present invention may employ various shaped tubes, sheaths, and the like, which may carry out a variety of functions. In addition, those skilled in the art will appreciate that the present invention may be practiced in any number of contexts and that the illustrative embodiment as described herein is merely one exemplary application for the invention. For example, the present invention may be applicable to various types of animals and other applications that require precise positioning of devices or drugs. Further, such general techniques that may be known to those skilled in the art are not described in detail herein.

The present system avoids the passive transportation of any pyrogens, bacteria, virus, toxins, or other substances. Thus, the sub-cutaneous or deep anatomic locale is kept cleaner, which can reduce clinical infection rates. Such a delivery system is beneficial to patient health and to reduce healthcare costs.

In addition, the system and methods of the present invention may use slip enhanced PTFE, and as such, while not requiring lubrication, actually performs better without any such foreign lubricious gels. This further maintains and promotes a clean, sterile, procedural, surgical site or wound Fewer variable substances at the surgical site may proportionately reduce the medical complications currently experienced in some procedures and specialties. Thus, the present invention is more efficient in the operating room for medical device placement than previous systems.

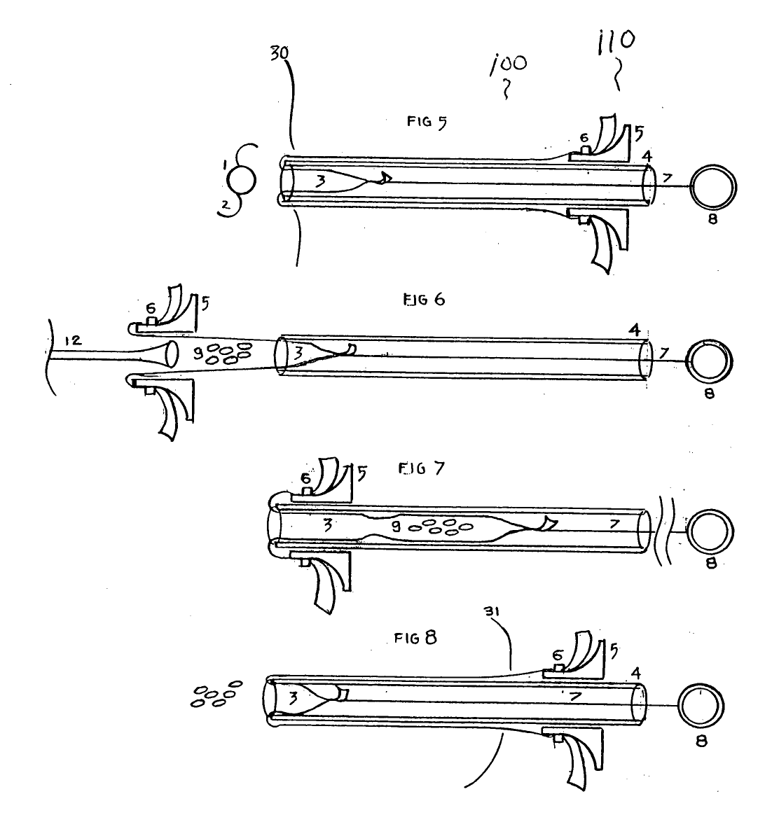

With reference to FIG. 4, in general, a delivery assembly 100 comprises a membrane sheath 3, a tube 4, and a guide assembly 110. In accordance with one aspect of the present invention, and with momentary reference to FIG. 5, in use, tube 4 along with membrane 3 is pushed through guide assembly 110, and a medical device such as the illustrated IOL lens 1 is deposited on or in a location of the human body such as an eye 30, a body cavity or channel (not illustrated in FIG. 5), or other body location.

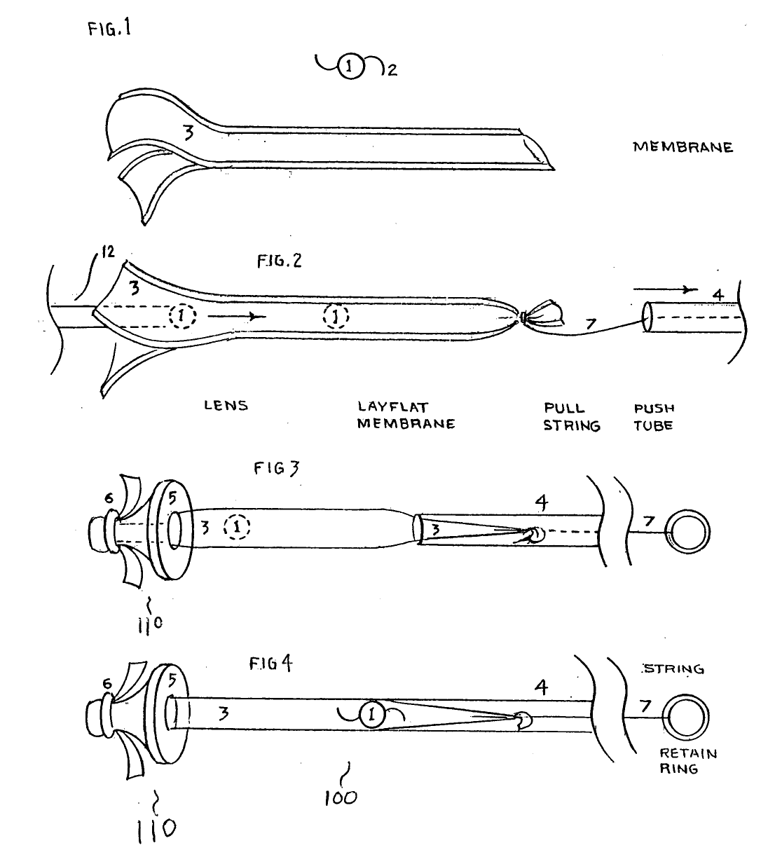

Referring now to FIG. 1, a membrane sheath 3 in accordance with one aspect of the present invention is shown. Membrane sheath 3 suitably comprises a thin, flexible polymeric substrate such as Memcath's™ slip enhanced Generation II PTFE film. However, membrane sheath 3 may suitably comprise other similarly performing polymer substrates such as fluorinate ethylene propylene (FEP), perfluoroalkoxy (PFA), other PTFE films, and the like. Advantageously, membrane 3 has sufficient lubricity to smoothly slide out of and over the exterior of, for example, a tube 4 as illustrated in FIGS. 2-5. Thus, any suitable material having sufficient slip, strength, integrity, flexibility and lubricity may be utilized in accordance with the present invention to form membrane 3, provided the material has sufficient strength and flexibility to be medically acceptable when in use.

In accordance with various aspects of the present invention, membrane 3 comprises a polytetrafluoroethylene resin, a modified PTFE resin, or combinations thereof. In accordance with one aspect of the present invention, membrane 3 is formed from a sintered PTFE film formed by skiving it off a billet to a thickness of less than 0.005 in. PTFE billet may comprise a modified PTFE, such as Hoechst TFM 1700 or TFM 1702 or other chemical compound available from DeWall Industries of Saunderstown, R.I. under the names DW/200, and DW/220 respectively or other processors. Such material comprises a modified PTFE polymer, modified by the addition of a small amount of perfluoro propyl vinyl ether (PPVE). It is believed that the addition of PPVE causes the PTFE to be more amorphous and more plasticized than pure crystalline PTFE. Such modification also permits the film to be heat sealed upon itself (i.e., interfacial fusion), in accordance with various aspects of the present invention.

In accordance with a further aspect of the present invention, membrane 3 having multiple global sources may also comprise a modified PTFE resin available from DuPont under the name Mitsui-DuPont TG 70-J which has been sintered into billets, annealed, and skived to a thickness of on the order of 0.001 in. Additionally, it should be appreciated that other PTFE films may be suitably used as may be now known or hereafter devised by those skilled in the art. For example, PTFE homopolymers or copolymers with comonomers like PPVE, PFA and the like may be suitably used. It is important, however, that the film be usable to form membrane 3 which when used in connection with tube 4 can be easily withdrawn, (i.e. does not “lock”) when membrane 3 is (inverted) withdrawn in a non-lubricated or “dry” state. The membrane materials useful in accordance with the present invention also have use in connection with various designs, such as those described in U.S. Pat. No. 5,531,717, issued Jul. 2, 1996, U.S. Pat. No. 5,676,688, issued Oct. 14, 1997, and U.S. Pat. No. 6,240,968, issued Jun. 5, 2001, the descriptions contained in each of those references are hereby incorporated herein by reference.

In accordance with various aspects of the present invention, membrane sheath 3 has a thickness on the order of less than 0.005 inches thick. It should be appreciated, however, that membrane sheath 3 may have thickness in excess of 0.005 inches. In accordance with one embodiment of the present invention, membrane sheath has a thickness less than 0.001 inches. The polymer membrane sheath may be made of a substrate of various thickness. As will be described in detail below, membrane sheath 3 may be used to place an intraocular lens 1 with anchoring and positioning haptics 2 into an eye.

With reference to FIG. 2, an IOL 1 is illustrated being loaded into a membrane sheath 3. As will be appreciated, IOL 1 may be loaded in a variety of ways. In accordance with one aspect of the present invention, IOL 1 may be positioned in membrane sheath 3 via a plunger/pusher rod 12. The membrane sheath 3 may deploy the medical device or drug to the desired position in the body where the medical device or drug was inserted, positioned, and enveloped into the membrane sheath. For example, three centimeters from the leading edge into the sheath equals three centimeters depth into the body.

One end of the membrane sheath 3 may be attached to a thread 7 (e.g., cotton, floss, nylon, and the like), which is strung through a pusher tube 4, made of a semi-rigid material such as polyvinylchloride (PVC), polycarbonate (PC), acyrlonitrile butadiene styrene (ABS), nylon, and the like. The pusher tube may be made from a clear material such that the surgeon or other operator of the pusher tube can easily monitor the travel position of the device.

With reference to FIG. 3, lens 1 is shown positioned in membrane 3. Lens 1 may be suitably deposited into an eye by use of a pusher tube 4. As will be appreciated, prior to depositing lens 1, membrane 3 along with lens 1 may be loaded into pusher tube 4 in a variety of ways. In accordance with one aspect of the present invention, membrane 3 may be pulled into the pusher tube 4 by the string 7. A suitable retaining ring 8 may be attached to string 7 to facilitate pulling membrane 3 into pusher tube 4. In accordance with another aspect of the present invention, membrane sheath 3 collapses and protects the medical device such as an intraocular lens (IOL) or a stent, as the sheath is pulled into the tube 4. With momentary reference to FIG. 4, when loaded into pusher tube 4, membrane 3 is interposed between pusher tube 4 and lens 1 such that lens 1 is only in contact with membrane 3.

In accordance with one embodiment of the present invention, a guide assembly 110 such as guide ring 5 is secured to the end of membrane 3 away from string 7. Guide ring 5 may be secured by a snap or twist ring 6 to membrane 3. FIG. 4 shows the lens 1 in the delivery assembly 100 ready for deployment.

With momentary reference to FIG. 5, as shown, in use of assembly 100, lens 1 may be deposited into location in an eye 30. The inverting sheath deployment delivery system has the versatility to accommodate multiple medical applications with dimensional changes, to accommodate various diameters and lengths. During use of assembly 100, membrane 3 is pushed through tube 4 and inverted (i.e., folded over) such that the membrane is inverted over the outside of tube 4. While the way in which membrane 3 can be inverted may vary, in accordance with one aspect of the present invention, membrane 3 is inverted through the use of the secure connection between membrane 3 and guide assembly 110. With reference to FIGS. 4 and 5, for example, an end of membrane 3 is connected to guide assembly 100, such as through the use of any snap or twist ring 6. As the tube, along with the membrane, passes through the guide assembly, one end of the membrane is secured to guide assembly 110. In this manner, the membrane 3 may be unfolded over the outside of tube 4 such that membrane 3 is interposed between tube 4 and the eye 30. It will be appreciated that, in this manner, lubricant is not delivered and lubricant does not remain in the eye during IOL delivery.

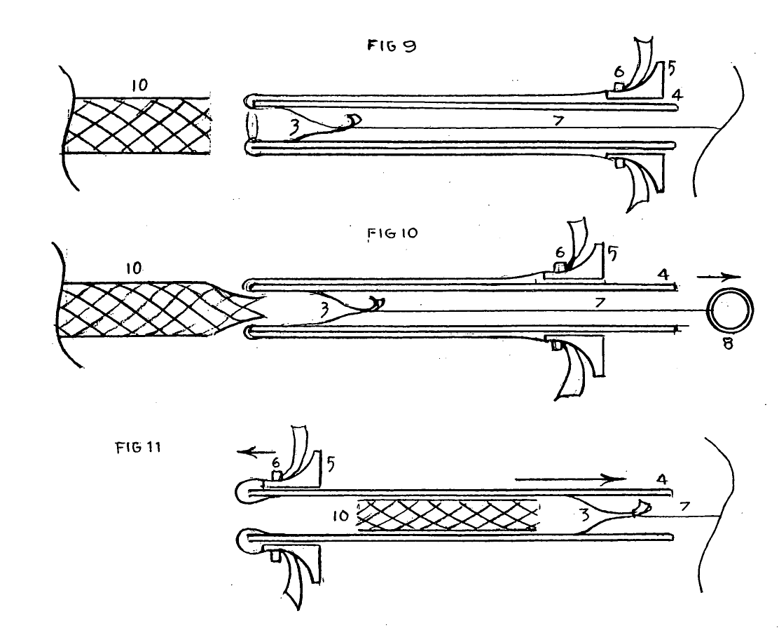

In accordance with another aspect of the present invention, FIG. 6 shows the loading of a drug such as radiologic seeds 9 used for prostate and bladder cancer therapies. Radiologic seeds 9 may be suitably deposited into an affected cancerous site by use of pusher tube 4. As will be appreciated, prior to depositing radiologic seeds 9, membrane 3 along with seeds 9 may be loaded into pusher tube 4 in a variety of ways. In accordance with one aspect of the present invention, membrane 3 may be pulled into the pusher tube 4 by the string 7. A suitable retaining ring 8 may be attached to string 7 to facilitate pulling membrane 3 into pusher tube 4. Seeds 9 may be positioned in membrane 3 by a variety of ways including using a suitable plunger 12 to locate/position the seeds in the membrane sheath. With momentary reference to FIG. 4, when loaded into pusher tube 4, membrane 3 is interposed between pusher tube 4 and seeds 9 such that seeds 9 are only in contact with membrane 3. FIG. 7 shows the drug seeds in position ready for deployment.

With momentary reference to FIG. 8, as shown, in use of assembly 100, seeds 9 may be deposited into the affected cancerous site, through a body orifice 31 such as the urethral meatus. Alternatively, the body orifice 31 could be a surgical, scalpel created access port such as might be required for tumors at or beneath the epidermal layers. During use of assembly 100, membrane 3 is pushed through tube 4 and inverted (i.e., folded over) such that the membrane is inverted over the outside of tube 4. In this manner, the membrane 3 may be unfolded over the outside of tube 4 such that membrane 3 is interposed between tube 4 and the patient's body. FIG. 8 shows the seeds/drug deposited into the affected cancerous site. It will be appreciated that, in this manner, contaminants from the conjunctiva, urethra, or other body part or channel are not dragged into the wound during introduction of the medical device and/or drugs.

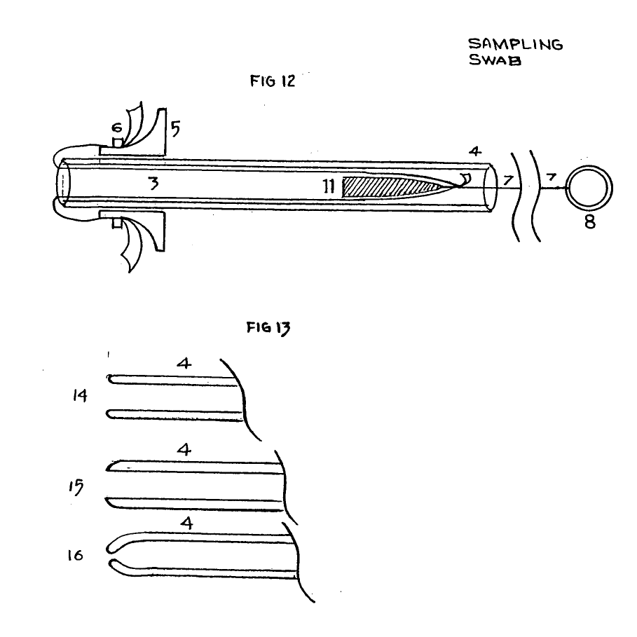

In accordance with another aspect of the present invention, FIG. 9 shows a stent 10 ready to be loaded into the assembly. FIG. 10 shows stent 10 being drawn down (collapsing) into the membrane 3 as the loading string 7 is pulled back. In some cases depending on the stents' geometry, and there are many (e.g. flared points at each end), a funnel may be needed to help position the stent 10 to properly collapse into the membrane 3. FIG. 11 shows the collapsed stent 10 in position in the membrane 3 and ready for deployment.

In accordance with another aspect of the present invention, the membrane sheath will advance with a sampling reagent, and the sampling reagent will then retreat back into the membrane sheath, thus providing a completely sterile, localized bacterial sampling for pathology diagnostic use. In the context of this embodiment of the present invention, FIG. 12 shows a textile or sponge swab 11 attached to the tied end of membrane 3, wherein swab 11 is enveloped inside the membrane 3. In this embodiment, the swab would be advanced to the site. The whole assembly would be twisted, rubbed against the patient's tissue in question and then the assembly would be withdrawn and simultaneously re-inverted with the pull/loading string to the original pre-deployment position shown in FIG. 12, thus isolating the swab. The assembly may then be polybagged and sent to the pathology lab for analysis.

In accordance with various aspects of the present invention, FIG. 13 shows three exemplary pusher tube 4 tips 14, 15, 16. Tip 14 comprises a pusher tube 4 with straight tips 14 with a constant inner diameter/outer diameter (ID/OD) radii. Tip 15 comprises a simple outer diameter beveled corner break and tip 16 comprises a pusher tube with a tapered tip with ID/OD both reduced at the proximal end.

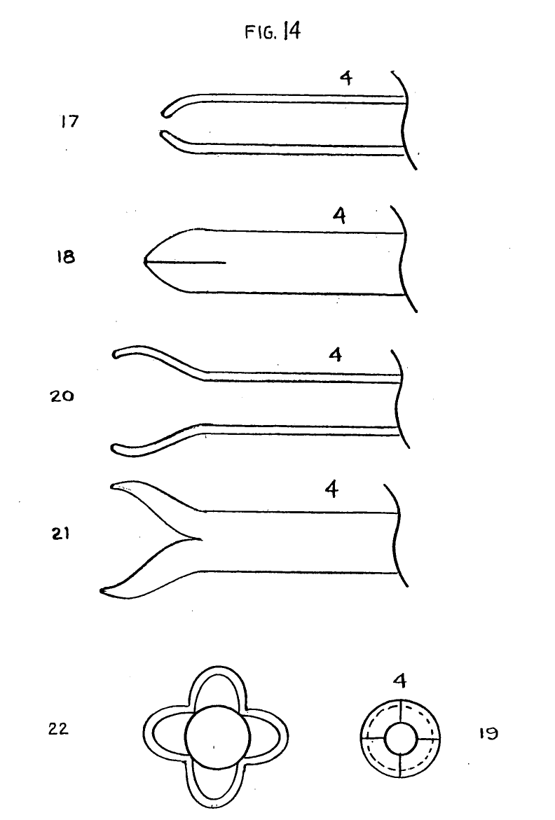

FIG. 14 shows an alternate tapered tip 17, 18, 19 with two or more slots allowing a semi-rigid (PVC) pusher tube 4 to flower open as a device passes through the tube 4, to which the tip inner diameter is smaller than the main tube body's inner diameter. This permits ease of entry into the body, with a tapered tip, yet allows a larger diameter medical device than the tapered inner diameter opening to pass through when the membrane 3 elsewhere shown is deployed. FIG. 14 shows the slotted tip in the flowered open position 20, 21, 22.

FIG. 15 shows an alternate pusher tube 23 with various alternative distal ends 24 and 24A. This will help to backload the IOL in contrast to the front-loading technique discussed above.

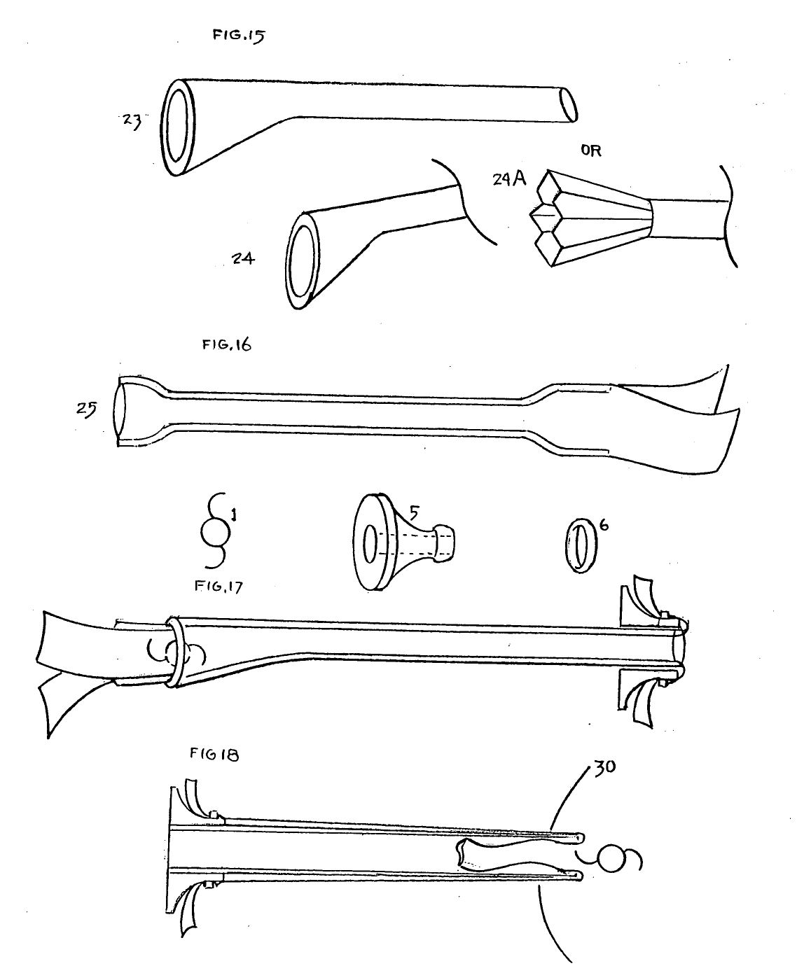

FIG. 16 shows an alternate membrane sheath 25 that can function with an IOL. The IOL 1, the guide rings 5, and the snap-retaining ring 6 are as discussed in detailed above.

FIG. 17 shows an exemplary back load assembly version. The IOL 1 may be shipped in this position.

FIG. 18 shows the deployment of an IOL into an eye 36. The expanded proximal end of the pusher tube has collapsed as the guide ring moved into final position.

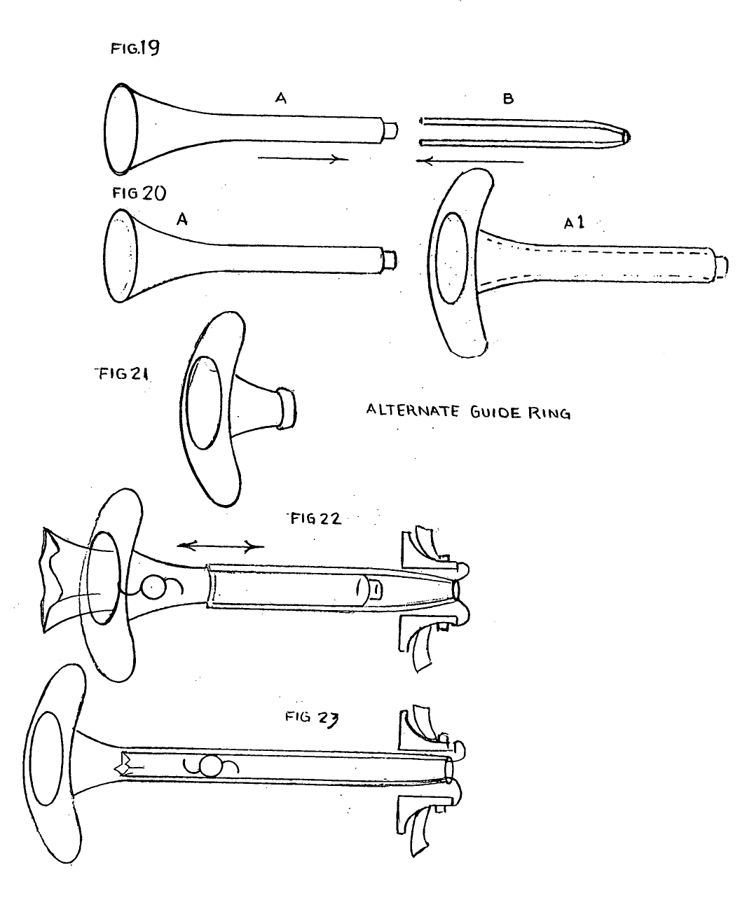

In accordance with another embodiment, FIG. 19 shows two tube sections. The tube sections will slide together in operation (see FIG. 20), and then later slide and rest together for loading of the membrane, assembly of the guide ring/snap and loading of the IOL. FIGS. 22 and 23 show the IOL in deployment ready mode.

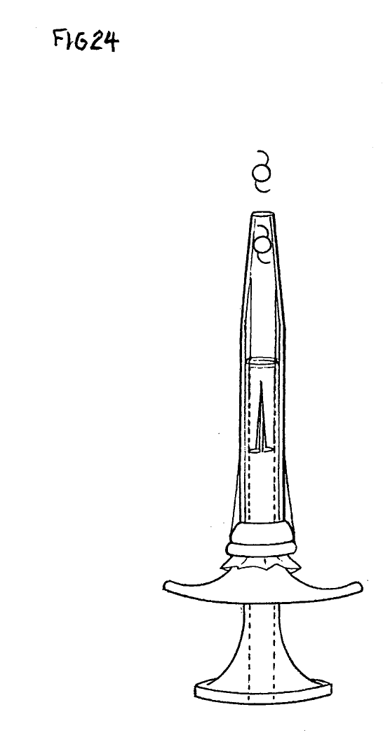

FIG. 20 shows an alternate distal end A1 which will butt up against the palm of the surgeon's hand. FIG. 21 shows an alternate guide ring with finger grips. These two flanges, the end A1 of the pusher tube illustrated in FIG. 20 and the finger grip illustrated in FIG. 21 will allows the surgeon to squeeze and deploy the device ready (see FIG. 23) to deployment executed (see FIG. 24).

The present invention has been described above with reference to an exemplary embodiment. However, those skilled in the art will recognize that changes and modifications may be made to the exemplary embodiment without departing from the scope of the present invention. For example, the various processing steps dictated by the present invention, as well as the components for carrying out the processing steps, may be implemented in alternate ways depending upon the particular application or in consideration of any number of cost functions associated with the operation of the system. These and other changes or modifications are intended to be included within the scope of the present invention.

What is claimed is:

1. A delivery assembly for passively delivering a medical device, the assembly comprising:
a tube having first and second open ends;
a polytetrafluoroethylene membrane sheath having a first end and a second end, wherein the first end of the membrane sheath is disposed inside the tube, the second end of the membrane sheath is disposed exterior of the tube, and the medical device is positioned on the membrane sheath inside the tube such that the membrane sheath is interposed between the medical device and the tube; and
a guide assembly connected to the second end of the membrane sheath, wherein the guide assembly is disposed exterior the tube;
wherein the second end of the membrane sheath is inverted over the second open end of the tube.

2. The delivery assembly of claim 1, wherein the medical device comprises an intraocular ophthalmic lens.

3. The delivery assembly of claim 1, wherein the medical device comprises a stent.

4. The delivery assembly of claim 1, wherein the medical device comprises a swab.

5. The delivery assembly of claim 1, wherein the medical device comprises a medical drug for delivery into a body cavity.

6. The delivery assembly of claim 1, further comprising a string attached to the first end of the membrane sheath, wherein the string extends through the first end of the tube.

7. The delivery assembly of claim 1, wherein the second open end of the tube has a straight tip.

8. The delivery assembly of claim 1, wherein the second open end of the tube has a tapered tip.

9. The delivery assembly of claim 8, wherein the tapered tip has a plurality of slots provided longitudinally along the length of the tip.

10. The delivery assembly of claim 1, wherein the second open end of the tube has a beveled corner tip.

11. The delivery assembly of claim 1, wherein the tube and the membrane sheath are not lubricated.

12. A delivery system for insertion into an anatomical canal, the delivery system comprising:
a tube having first and second open ends;
an unfolding polytetrafluoroethylene membrane sheath having a first end and a second end, wherein the first end of the membrane sheath is disposed inside the tube and the second end of the membrane sheath is disposed exterior of the tube;
a medical device pre-loaded inside the tube, wherein the medical device is positioned inside the membrane sheath inside the tube; and
a guide assembly connected to the second end of the membrane sheath;
wherein the second end of the membrane sheath is inverted over the second open end of the tube, and wherein the membrane sheath is suitably removed from the second open of the tube such that the membrane sheath is interposed between the medical device and the anatomical canal as the medical device is inserted into the anatomical canal.

13. The delivery system of claim 12, wherein the medical device comprises an intraocular ophthalmic lens.

14. The delivery system of claim 12, wherein the medical device comprises a stent.

15. The delivery system of claim 12, wherein the medical device comprises a swab.

16. The delivery system of claim 12, wherein the medical device comprises a medical drug for delivery into a body cavity.

17. A method for delivery of a medical device onto a delivery location of a patient, comprising the steps of:
providing a delivery assembly comprising a tube having first and second open ends, a polytetrafluoroethylene membrane sheath having a first end and a second end, wherein the first end of the membrane sheath is disposed inside the tube, the second end of the membrane sheath is disposed exterior of the tube, the medical device is positioned on the membrane sheath inside the tube, the second end of the membrane sheath is inverted over the second open end of the tube and a guide assembly connected to the second end of the membrane sheath, wherein the guide assembly is disposed exterior the tube;
inserting the second end of the tube into an anatomical canal of a patient;
maintaining the first end of the tube outside of the anatomical canal of the patient;
pushing the tube into the anatomical canal of the patient until the second end of the tube reaches the delivery location for the medical device, thereby causing the membrane sheath to be withdrawn, further unfold and be interposed between the tube and the patient's anatomical canal; and
extracting the membrane sheath and the tube from the patient's anatomical canal after the medical device has been introduced onto the delivery location in the patient.
